(19) United States
(12) Patent Application Publication
(10) Pub. No.: US 2003/0181353 A1
(43) Pub. Date: Sep. 25, 2003

(54) Composition & use as analgesic, anti-inflammatory, wound healing agent, for treatment of heart conditions, assessment of heart function & tissue & cell protection & healing & reperfusion, mood disorders & symptoms & sequelae of menopause & for inducing unconsciousness, sleep & anesthesia

(76) Inventor: Jonathan W. Nyce, Titusville, NJ (US)

Correspondence Address: HOWREY SIMON ARNOLD & WHITE, LLP, BOX 34, 301 RAVENSWOOD AVE., MENLO PARK, CA, 94025, US

(21) Appl. No.: 10/349,219

(22) Filed: Jan. 21, 2003

Related U.S. Application Data
(62) Division of application No. 09/762,090, filed on Feb. 1, 2001, now abandoned.
Application No. 09/762,090 is a 371 of international application No. PCT/US99/17642, filed on Aug. 3, 1999.
(60) Provisional application No. 60/095,090, filed on Aug. 3, 1998.

(51) Int. Cl. A61K 31/00

(52) U.S. Cl. 514/1

(57) ABSTRACT

Composition and formulations comprise a brain, heart, lung and/or vascular system protecting amount of a first agent such as folinic acid, pharmaceutically acceptable salts thereof or mixtures thereof and a carrier, and optionally a second agent such as analgesics, heart medicines, anti-inflammatory agents, soporifics, muscle relaxants, anti-pyretic agents, anti-fibrillation agents, heart, brain, lung and vascular drugs, anxiolytic agents, mood controlling agents, and many others. The products are suitable for the treatment of previous mood disorders, symptoms and sequelae of menopause, pain, inflammation, insomnia, restless sleep, trauma, surgery, burns, conditions and diseases which bring these symptoms, ischemia, Supra Ventricular Tachycardia (SVT), heart conditions and heart failure, Acute Respiratory Distress Syndrome (ARDS), COPD, allergic rhinitis, and other conditions, and for reducing the number and severity of heart attacks, as well as for alleviating other diseases associated with the heart, and more generally, for the assessment of heart function. An edible product is prepared with the agent of the invention.

### RELATED APPLICATIONS

This application is based on International Application No. PCT/US99/17642, filed Aug. 3, 1999, and published on Feb. 17, 2000 in English as International Publication No. WO 00/07566, which in turn claims priority of U.S. Provisional Application No. 60/095,090, filed Aug. 3, 1998; both by the same inventor as this application.

### BACKGROUND OF THE INVENTION

1. Field of the Invention

This invention relates to a composition and formulations comprising folinic acid, salts thereof or mixtures thereof and a carrier and, optionally, other therapeutic agents. The products are suitable for the treatment of various diseases and conditions such as sleeplessness, insomnia, inflammation, pain, ischemia, wounds, burns, mood disorders, symptoms and sequelae of menopause, and various heart diseases, for reducing the number and intensity of heart attacks and stroke, and for inducing unconsciousness, sleep and anesthesia. In addition, these products are useful for assessing heart function and for protecting, healing and reperfusing body tissues, among others. The agent is provided also as an edible product and kit for its preparation.

2. Description of the Background

Adenosine is a purine involved in intermediary metabolism. In addition, it participates in the regulation of physiological activity in a variety of mammalian tissues as well as in many local regulatory mechanisms, such as those occurring in synapses in the central nervous system (CNS) and at neuroeffector junctions in the peripheral nervous system. In addition, adenosine is also known to have effects in other systems. Examples of these are its action to suppress pacemaker activity and slow AV conduction, to possess antiarrhythmic and arrhythmogenic effects, modulate autonomic control and trigger the synthesis and release of prostaglandins, to have potent vasodilatory effects and modulate vascular tone. At present, adenosine is used clinically for the treatment of SupraVentricular Tachycardia (SVT) and other cardiac anomalies, as well as for testing cardiovascular function. Adenosine analogues also are being investigated for use as anti-convulsant, anxiolytic and neuroprotective agents. Moreover, adenosine also protects tissues subjected to ischemia (oxygen deprivation) and aids their reperfusion, e.g. brain following stroke or other acute or chronic brain ischemia-producing conditions and diseases, heart following heart attack or other acute or chronic heart ischemia-producing conditions or diseases, and other organs at risk for ischemia associated with diseases and condition processes, acute and chronic physiological events and in transplantable organs during the harvest and transportation stages prior to transplantation as well as in already transplanted organs, among others. Adenosine analogues also are being investigated for use as anti-convulsant, anxiolytic and neuroprotective agents. Adenosine is also a natural anti-inflammatory agent which, for example, is known to mediate the anti-inflammatory effect of methotrexate, and promotes and accelerates wound healing and regulates neutrophil function via activation of a serine/threonine phosphatase. In the CNS, adenosine is known to inhibit the release of neurotransmitters, such as acetylcholine, noradrenaline, dopamine, serotonin, glutamate, and GABA. Adenosine was also shown to depress neurotransmission itself, to induce spinal analgesia possibly by reducing neuronal firing, and to possess anxiolytic properties. Although adenosine has various therapeutic applications as described above, it has an extremely short half life (about a second). Adenosine's short half life and its propensity to cause angina-like pain make it a poor choice for therapeutic applications.

Folinic acid is an intermediate product of the metabolism of folic acid, and is believed to be the active form into which folic acid is converted in the body. It is also known that ascorbic acid or Vitamin C is necessary for the conversion of folic acid to folinic acid. Folinic acid has been used therapeutically as an antidote to folic acid antagonists such as methotrexate which block the conversion of folic acid into folinic acid. Folinic acid also has been used as an anti-anemic, because of its ability to combat folate deficiency. Folinic acid has heretofore never been used in patients afflicted with adenosine depletion nor in a method to therapeutically elevate adenosine levels in the brain, heart, or other organs.

Mood disorders such as clinical depression are quite different from the blues everyone feels at one time or another and even from the grief of bereavement. They are more debilitating and dangerous, and in many cases an overwhelming sadness combines with a number of other symptoms. Some people become preoccupied with suicide, while others experience extreme highs that lead to dangerous behavior, and many are plagued by guilt and a sense of worthlessness. Some often have difficulty thinking clearly, remembering, or taking pleasure in anything. They may feel anxious and sapped of energy and have trouble eating and sleeping or may, instead, feel exhilarated and want to eat and sleep excessively. The prevalence of various types of mood alterations is surprisingly high. It is estimated that 5 to 12% of men and 10 to 20% of women in the U.S. will suffer from a major depressive episode at some time in their life. Roughly half of these individuals (about 1-1.5% of Americans) will become depressed more than once, and up to 10% (about 1.0 to 1.5% Americans) will experience manic phases, a condition known as manic-depressive illness or bipolar disorder, in addition to depressive ones. Mania is marked by a decreased need for sleep, rapid speech, delusions of grandeur, hyperactivity and a propensity to engage in such potentially self-destructive activities as promiscuous sex, spending sprees or reckless driving. Beyond the pain and disability mood disorders bring, they are also potential killers. Statistics show that as many as 15% of those who suffer from depression or bipolar disorder commit suicide each year. In 1996 the Centers for Disease Control and Prevention listed suicide as the 9th leading cause of death in the U.S. only slightly behind infection with the AIDS virus, taking the lives of 30,862 people. This number, however, is believed to be underestimated, since at least some fraction of deaths attributed to other diseases and to automobile accidents may be concealed suicides.

Considerable evidence indicates that regardless of the initial triggers, the final common pathways to depression involve biochemical changes in the brain. It is these changes that ultimately give rise to deep sadness and the other salient characteristics of depression. The full extent of those alterations is still being explored, but in the past few decades, and especially in the past several years, efforts to identify them have progressed rapidly. Overall psychotherapy and anti-depressants are currently being used, while other treatments such as electroshock have fallen from favor. Although today's antidepressants have fewer side effects than those of old and may be extremely helpful in many cases, depression continues to exact a huge toll in suffering, lost lives and reduced productivity. Geneticists have provided some of the oldest proof of a biological component to depression in many people. Depression and manic-depression frequently run in families. As geneticists continue searching for the culprit genes, others are concentrating on neurochemical pathways and particularly on neurotransmitters. More specifically, many cases of depression apparently stem at least in part from disturbances in brain circuits that convey signals through certain neurotransmitters of the monoamine class. These biochemicals, all derivatives of amino acids, include serotonin, norepinephrine and dopamine. Of these, only evidence relating to norepinephrine and serotonin is abundant. In addition, there is also evidence for the involvement of dysregulation of the hypothalamic-pituitary-adrenal (HPA) axis, the system that manages the body's response to stress. When a threat to physical or psychological well-being is detected, the hypothalamus amplifies production of corticotropin-releasing factor (CRF), which induces the pituitary to secrete ACTH. ACTH then instructs the adrenal gland atop each kidney to release cortisol. Together all the changes prepare the body to fight or flee and cause it to shut down activities that would distract from self-protection. For instance, cortisol enhances the delivery of fuel to muscles. At the same time, CRF depresses the appetite for food and sex and heightens alertness. A chronic activation of the HPA axis, however, may lay the ground for illness and possibly for depression. Notably, study after study has shown an elevation of CRF concentrations in cerebrospinal fluid in depressed patients, when compared with control subjects or individuals with other psychiatric disorders. Moreover, the delivery of CRF to the brains of laboratory animals was observed to produce behavioral effects similar to those observed in depression in humans, namely, insomnia, decreased appetite, decreased libido and anxiety. Accumulating findings indicate that severe depression also heightens the risk of dying after a heart attack or stroke. And it often reduces the quality of life for cancer patients and might reduce survival time.

The heart is a four-chambered pump which controls blood flow through a remarkable series of valves, which open and close at just the right moment in each cycle of the heartbeat. For most persons, these valves function flawlessly during their entire lives. In a small percentage of the population that experiences heart valve problems, the most common is mitral valve prolapse, a condition more frequently encountered in women than men. The mitral valve controls blood flow between the left upper and lower chambers of the heart. The upper chamber (the left atrium) receives freshly oxygenated blood from the lungs and delivers it, through the mitral valve, to the left ventricle (the heart's main pumping chamber). The contraction of the left ventricle, in turn, sends fresh blood coursing through the arterial system to provide oxygen and nutrients to the entire body. In mitral valve prolapse, one or both of the heart valves are enlarged, and the strings of connective tissue are too long or too stretchy, permitting the valve to be pushed upward into the atrium during contraction of the ventricle. In more severe cases, an improperly closed valve permits some blood to leak backward into the atrium, i.e. mitral valve regurgitation. Generally, however, mitral valve prolapse is not serious enough to cause much of a problem. If the condition does produce symptoms, they most often take the form of heart palpitations or “skipped beats”, or other heart rhythm irregularities. The next most frequent problem is chest pain, shortness of breath or a tendency to tire readily.

Statistics show that 35% of Americans die of heart disease. Risk factor characteristics found in people who are prone to heart problems are age, family history, gender, weight, smoking, cholesterol levels, and diabetes. Heart disease is more common in the elderly and diagnosed most often between ages 45 and 55. Chances increase if a parent had heart disease before age 50. Men are more likely than women to develop heart disease before age 50. After that, a woman's chance of having heart problems are just as great. If you weigh 20% above what is normal for your height, and carry your excess weight around your middle, your likelihood of having heart disease increases. Smokers are at twice the risk of developing heart problems, and even slightly higher than normal blood pressure doubles the risk of stroke. A cholesterol count of less than 200 is desirable, between 200 and 240 is borderline, and greater than 240 is high, and finally diabetics may be twice as prone to heart disease, or may develop problems at a younger age.

Heart attacks occur when the blood supply to a portion of the heart muscle (myocardium) is severely reduced or stopped, that is when one of the arteries that supplies blood to the heart muscle (coronary arteries) is obstructed or blocked. This blockage may be due to arteriosclerosis (a build-up of deposits of fat-like substances), a blood clot (coronary thrombosis), or a coronary vessel spasm coupled with a near total obstruction. If the blood supply to a portion of the heart stops, that portion will no longer receive the oxygen or nutrients necessary to carry out its function and will die. If a large enough portion is damaged, irreversible damage may result, leading to death. The pain of a heart attack is a severe, sharp, piercing pain, which results from heart tissue ischemia (decreased blood supply). It is normally felt as a heavy weight on the chest, but may radiate to the neck, jaw, one or both arms, and between the shoulder blades. These symptoms usually last for long periods of time and are not relieved by nitroglycerin, as are the palpitations or “skipped beats”, or other heart rhythm irregularities. The next most frequent problem is chest pain, shortness of breach or tendency to tire readily. The nation's longest-running heart study suggests that about one heart attack in four produces no symptoms, or at least none that the victim associates with a heart problem. These so-called “silent heart attacks,” however, are only the most extreme case of a still more prevalent condition called “silent ischemia,” a chronic shortage of oxygen- and nutrient-bearing blood to the heart. Both conditions put their victims at significant risk. The cause of ischemia, silent or otherwise, is in many instances arteriosclerosis, the progressive narrowing of the heart's arteries from accumulations of cholesterol plaque. This reduction in blood supply generates a protest from the heart, the crushing pain called angina. But in perhaps 25 to 30% of heart attack victims, there were no previous symptoms of these gradually developing blockages. The Framingham heart study, which has followed 4,000 Massachusetts men for more than 40 years, has found that 25% of their subjects' heart attacks go unnoticed until their annual EKGs detect their after-effects. The absence of pain, however, doesn't mean an absence of damage. The heart has a built-in reserve capacity, allowing it to suffer a certain amount of scarring and weakening from a heart attack and continue to meet the body's needs. But further ischemia or another heart attack, even a mild to moderate one, may prove fatal, because of a lack of reserve capacity. Even those who survive another heart attack are at increased risk of becoming cardiac cripples, disabled by congestive heart failure or arrhythmias, i.e. heartbeat irregularities. There is no way of predicting absolutely who is a candidate for silent ischemia, but, statistically, the greater the number of risk factors for coronary artery disease, the more likely to be a candidate. Those risk factors include some that may not be controlled, e.g. age, sex and genetic predisposition to arteriosclerosis, and those that may be influenced, like diabetes, high blood pressure, high blood cholesterol, smoking, lack of exercise, and obesity. The screening for undetected ischemia is screened for by means of a medical history and physical examination and a cardiac stress test: a workout on a treadmill while the heart function is monitored.

Sudden cardiac deaths sometimes occur in patients with no anatomical or electrophysiologic evidence of heart disease. In these patients silent (asymptomatic) ischemia from coronary artery spasm may be the cause of cardiac arrest by triggering ventricular fibrillation. Silent myocardial ischemia usually occurs in patients with known fixed coronary artery disease. In patients with stable angina and those who have had myocardial infarction, silent myocardial ischemia is a frequent occurrence and is associated with an increased risk of cardiac events, including sudden death. However, silent myocardial ischemia may also occur in patients with normal coronary arteries, and it may be severe enough to elevate ST segments and trigger serious arrhythmias. The presence of spontaneous ST-segment elevation along with serious arrhythmias in patients with variant angina many times carries an ominous prognosis. Ventricular fibrillation is a condition in which disordered electrical activity causes the ventricles to contract in a rapid, unsynchronized, uncoordinated fashion. When this occurs, little or no blood is pumped from the heart. Collapse and sudden death follows unless medical help is provided immediately. If treated in time, ventricular tachycardia and ventricular fibrillation can be converted into normal rhythm with electrical shock. Rapid heart beating can be controlled with medications by identifying or destroying the focus of rhythm disturbances. These days one effective way of correcting these life-threatening rhythms is by using an electronic device called an implantable cardioverter-defibrillator. Subsequent studies demonstrated that patients with ventricular fibrillation were sometimes found to have no flow-limiting coronary artery lesions, no angina pectoris, no prior myocardial infarction (MI), and no structural abnormalities of the heart that could account for cardiac arrest. However, frequently they have had transient episodes of dynamic ST-segment changes associated with spontaneous or ergonovine-induced coronary artery spasm. In many, reperfusion, rather than ischemia itself, correlates with the onset of the arrhythmia. Reperfusion-induced arrhythmias may be due to calcium overload following ischemic injury, which occasionally results in cardiac arrest while jogging, which comes as no surprise since both silent ischemia and coronary spasm may be induced during exercise. According to some clinicians, even in the absence of earlier chest pain and fixed coronary artery disease, transient ischemia due to coronary spasm should be considered in the diagnostic evaluation of survivors of cardiac arrest. In these patients, myocardial ischemia can be severe enough to trigger life-threatening arrhythmias. Anti-ischemic therapy may protect against the arrhythmia in patients with silent (or asymptomatic) ischemia and ventricular arrhythmia. Patients with ventricular tachycardia secondary to myocardial scarring usually require additional antiarrhythmic therapy, pharmacologic or nonpharmacologic.

A stroke, also called a “brain attack”, happens when brain cells die because of inadequate blood flow. A brain attack occurs when a blood vessel bringing oxygen and nutrients to the brain bursts or is clogged by a blood clot or some other particle. Because of this rupture or blockage, part of the brain doesn't get the flow of blood it needs. Deprived of oxygen, nerve cells in the affected area of the brain can't function and die within minutes. And when nerve cells can't function, the part of the body controlled by these cells can't function either. The devastating effects of stroke are often permanent because dead brain cells aren't replaced. Strokes kill about 150,000 Americans each year and are the leading cause of adult disability.

Paroxysmal supraventricular tachycardia (SVT) is the most common sustained cardiac arrhythmia in pregnant women. Because nearly 50% of these supraventricular tachyarrhythmias fail to respond to vagal maneuvers, other therapies are used, including electrocardioversion and pharmacologic agents. Propranolol, verapamil, and adenosine have Food and Drug Administration-approved labeling for acute termination of supraventricular tachycardia. Verapamil has been the most commonly used agent in the general population but it has several shortcomings, such as its potential to cause or exacerbate systemic hypotension, congestive heart failure, bradyarrhythmias, and ventricular fibrillation. In addition, verapamil readily crosses the placenta and has been shown to cause fetal bradycardia, heart block, depression of contractility, and hypotension. In addition to its effects on tachyarrhythmias, adenosine has been shown experimentally to reduce reperfusion injury following coronary ischemia and to reduce infarct size and prevent the “no-reflow phenomenon.” Thus, it may useful for protecting the heart during evolving MI. Adenosine has been used in the noninvasive evaluation of myocardial ischemia because of its ability to increase coronary blood flow. The uptake and redistribution of thallium is assessed following adenosine infusion, permitting identification of ischemic and damaged areas of the myocardium. Sensitivity, specificity, and predictive accuracy appear to be similar to those with exercise-stress testing or dipyridamole-mediated vasodilation. Finally, there is evidence that adenosine may be useful for seizures. Propranolol, verapamil, and adenosine have Food and Drug Administration-approved labeling for acute termination of supraventricular tachycardia (SVT). Verapamil has been the most commonly used agent in the general population but it has several shortcomings, such as its potential to cause or exacerbate systemic hypotension, congestive heart failure, bradyarrhythmias, and ventricular fibrillation. In addition, verapamil readily crosses the placenta and has been shown to cause fetal bradycardia, heart block, depression of contractility, and hypotension. Adenosine has several advantages over verapamil, including rapid onset, brevity of side effects, theoretical safety, and probable lack of placental transfer. Adenosine ultimately may prove to be the preferred agent for termination of paroxysmal supraventricular tachycardia (SVT) in the gravid woman as well. However, because it has an extremely short half life (about a second), and because of its propensity to cause angina-like pain, adenosine itself is a poor choice for the treatment of any of the above diseases and conditions as well as to counter the numerous physical and mental symptoms associated with mood disorders and conditions.

Clearly, given the high numbers of deaths involving myocardial disease, the possibility of identifying individuals who are at risk is of great importance, because an early detection permits an early treatment of the conditions. Electrocardiographic stress tests are used for this purpose while an individual exercises, but they lack high sensitivity and specificity. This is particularly the case with asymptomatic patients or with those with a typical thoracic chest pain of angina. In this case, in addition to the exercise stress test, cardiac perfusion images are also obtained with (rays, such as those emitted by ²⁰¹Th or ^{99m}Tc. A good number of coronary patients, however, cannot exercise at a level acceptable to validate the results of the test, such as those afflicted with severe arthritis and peripheral vascular diseases or conditions, among others. Hypertensive patients taking $-blockers and calcium channel antagonists also present a problem as to the detection of an adequate pulse and an effective stress test result while exercising. It is for these groups of patients who may not exercise adequately that pharmacological stress tests are most useful. In the United States about a third of patients referred for myocardial perfusion tests are administered pharmacological tests. For these, as well as for patients attended to in general practice, two kinds of drugs are utilized: coronary vasodilating drugs and positive inotropic agents.

Only two coronary dilating agents have been approved by the FDA for use in this test: dipyrimidol and adenosine, both of which dilate coronary arteries by elevating the level of adenosine in blood and increasing 4 or 5-fold the coronary blood flow. Once these changes are imparted, the patient is administered intravenously a radioactive agent, such as ²⁰¹Ta or ^{99m}Tc to do (-ray imaging. Although in a normal person the distribution of the radiolabel would be uniform, in a subject with one or more stenosis or occlusions in the coronary arteries will exhibit areas or “defects” in the artery (ies) irrigated by the radioactive label of different intensity (ies), which is attributable to ischemia or to myocardial necrosis. Contrary to those observed with exercise, the hemodynamic and electrocardiographic changes observed upon the administration of pharmacological agents like adenosine are slight. Usually the pulse will increase from 10% to 20% and the systemic arterial pressure from 5% to 10%, and the electrocardiographic depressions of the CT segments in the electrocardiogram (ECG) indicate a specific and serious sign of coronary artery disease. Thus, for many patients, the ability to undergo a pharmacological stress test is of extreme importance. However, many patients exhibit secondary effects (side effects), which in many cases result in severe bronchospasm, myocardial infarction and death. Thus, the administration of adenosine in a pharmacologic stress test is contraindicated in individuals afflicted with bronchoconstriction, asthma, including occult asthma, hypotension, and atrioventricular blockage of the second and third degrees. Many SVT patients and other subjects who would benefit from adenosine administration to assess their cardiovascular function, however, have hyper-responsive airways and are, thus, prone to bronchoconstriction in response to the administration of adenosine. This by itself, prevents them from being administered adenosine in order to avoid extreme bronchoconstriction, which may be life threatening.

In view of the foregoing, it is readily apparent that a large reduction in adenosine levels or adenosine depletion may lead to a broad variety of deleterious conditions in the CNS, the heart, and other tissues, and that the ability to treat, reverse and prevent its depletion is an extremely useful means of therapeutic intervention. An agent with a longer half life than adenosine would provide a great advantage for treatment of a variety of central nervous system (CNS) conditions such as mood disorders, including depression, schizophrenia, bipolar disease and other mood abnormalities, and for the treatment and control of symptoms and sequelae of menopause, as well as pain and inflammation. Moreover, the availability of an agent with a longer half life than adenosine, which at the same time is capable of preventing or reversing decreased adenosine levels or adenosine depletion would provide a great advantage for the therapeutic and prophylactic treatment of various diseases and conditions such as sleeplessness, sleep deprivation, insomnia and other sleep abnormalities, as well as for assessing heart function. In addition, such a composition would also be useful for inducing unconsciousness and anesthesia.

### SUMMARY OF THE INVENTION

This invention relates to the prevention and treatment of pain and inflammation, heart disease, CNS disorders, burns, wounds and traumatic injuries, to the induction on sleep and anesthesia, and the assessment of heart function. Examples of CNS mediated conditions are mood disorders, e.g., depression, schizophrenia, bipolar disease, compulsive obsessive disorder (COD), delirium, attention deficit disorder (ADD), overly aggressive behavior, and other mood abnormalities, as well as symptoms and sequelae of menopause, e.g. irritability and mood swings, which interfere with, and in many cases even prevent, a subject's daily functions, and significantly diminish his/her enjoyment of life. Examples of cardiovascular conditions are ischemia or hypoxia (oxygen deprivation), heart attacks, stroke, arrhythmias, SupraVentricular Tachycardia (SVT), ARDS, heart failure, and other disorders. These conditions benefit from the present treatment and other regardless of their origin, or whether they are accompanied by a decrease in adenosine levels or not, whether due to endogenous abnormalities or the result of exogenously administered substances. The composition may also be utilized to provide a “stress test” to assess the condition of a subject's heart. The present pharmaceutical composition comprises a carrier and a first agent selected from folinic acid, its pharmaceutically acceptable salts, and their mixtures, and optionally a surfactant and/or a second agent, such as other mood altering agents, anti-anxiolytic agents, nociceptics, sleep and anesthesia inducing agents, anti-inflammatory agents, hormonal agents, heart medications, diuretics, and analgesics, among others, and formulation ingredients suitable for administration by different routes. The first agent is generally present in the composition in an analgesic, anti-inflammatory, wound injury, or burn healing, mood stabilizing, sleep or anesthesia inducing, heart or vascular system protective amount, or in an amount effective for testing heart function.

Examples of other conditions for which the present treatment is effective are COPD, allergic rhinitis, emphysema, pulmonary vasoconstriction, chronic bronchitis, and hypertension, renal damage and failure produced by certain drugs and imaging substances, cancers, burns, sores and other tissue injuries, and the improvement of the quality of life of individuals in general, for whom other ethical drugs or substances have failed, and who are somewhat to fully incapacitated to conduct a normal life, and increase the likelihood that they will enjoy a happy and useful life. Secondary benefits also are an improvement of the quality of life in the individual's family as a result of health improvement, increased mobility, and the induction of prolonged mood elevation periods and restoration of the individual's outlook on life. The composition is provided in various formulations, in bulk or in unit form, and in the form of a foodstuff with other edible ingredients, e.g. energy bars, chewing gum, candy, drinks, cakes, mixed into salad, dressing, pasta, etc.

### DETAILED DESCRIPTION OF THE PREFERRED EMBODIMENTS

The present invention arose from a desire by the inventor to improve on prior therapies used for the prevention and treatment of mood disorders, either mono- or bi-polar, cardiovascular disease, e.g., heart disease, in particular when associated with oxygen deprivation, sleep deprivation, cat-napping, insomnia, acute and chronic pain and inflammation of any source, and for inducing unconsciousness and assessing heart function, among others. Many of the treatable conditions are associated with variations in adenosine levels or with adenosine depletion. The present treatment is effective, however, whether or not there is a marked adenosine reduction or depletion, and whatever its cause. Examples of the latter are genetic conditions, the administration of adenosine depleting drugs, deficient adenosine synthesis, decreased adenosine receptor sensitivity or neuronal transmission, high adenosine metabolism, and many other causes.

Adenosine is known to be a natural agent provided with heart, lung, kidney, and brain, particularly mood, stabilizing activities. However, because it has an extremely short half life (about a second), and because of its propensity to cause angina-like pain, adenosine itself is a poor choice for therapeutic use in the above listed diseases and conditions as well as to counter the numerous physical and mental symptoms associated with pain, inflammation, trauma, burns, oxygen deprivation, sleeplessness, and other diseases, and for testing heart function.

The inventor posited that an agent such as folinic acid, which is capable of causing the synthesis of adenosine, has a significantly longer half life than adenosine, and does not produce angina-like pain, would be better suited for administration to subjects afflicted with these and other conditions. Unexpectedly, through his research the present inventor found that folinic acid also has a sustainable mood and heart stabilizing and regulating activity, decreases pain and inflammation, whether associated with trauma, surgery, arthritis, or from other sources, induces unconsciousness, a-wave sleep and anesthesia, and is, therefore, useful for the treatment of pain, inflammation, skin lacerations, organ and tissue trauma, burns, insomnia, restless sleep, heart fibrillation, SVT, RDS, to alleviate the severity of heart attacks and stroke, chronic bronchitis, COPD, allergic rhinitis, auto-immune diseases, and a variety of other conditions and syndromes. He reasoned that, since folinic acid and its salts reach the skin, brain, and heart, and increase adenosine levels, they might have activities similar to adenosine. Thus, he proposed that folinic acid and its salts would be useful for treatment of the above described diseases. Later work not only confirmed his proposal, but in addition, showed folinic acid to be longer lasting than adenosine. Folinic acid and its salts are, thus, useful for treating mood alterations and disorders such as depression (mood elevating agent), bipolar disease, obsessive compulsive behavior, delusions, craziness, attention deficit disorder, and other mood pathologies. Folinic acid is also useful for treating mood alterations accompanying menopause and its sequelae, such as tiredness, sleeplessness and/or oversleeping, irritable behavior, depression, lack of appetite and/or excessive eating, early awakening from sleep and/or low a-wave sleep, lack of interest in life in general, and in sex in particular.

The inventor has shown that the administration of folinic acid induces de novo synthesis of adenosine in the heart and, when given orally, causes a dramatic increase in adenosine levels in the heart, lung and brain in an animal model. Since it has a more prolonged life than adenosine, and is not associated with the induction of angina-like pain, folinic acid represents an unexpected improvement over adenosine as an analgesic, anti-inflammatory, a heart medicine (e.g. as anti-fibrillator, anti-ischemic, and for the prevention and treatment of stroke, heart failure, heart attacks, SVT, ARDS, arrhythmias, etc.), for treating traumatic body injury, other heart, lung and vascular system pathologies, and for anxiety, it has sustained activity for inducing, prolonging and deepening sleep patterns and is, therefore, useful as a soporific (sleep inducing agent), and for the treatment and prevention of sleeplessness, sleep disorders, restless sleep, cat-napping, insomnia, early awakening from sleep, and low a-wave sleep patterns, as a mood controlling agent (mood elevation and flattening of high and low points of bipolar disease), for irritability control, and treatment of compulsive obsessive behavior (COB), paranoia, schizophrenia, depression, bipolar disease, and the like, for inducing anesthesia and for the assessment of heart function. Folinic acid, its salts and their mixtures are efficacious in the prophylaxis and treatment of these and other disorders and conditions where, for instance, increasing the level of adenosine is of therapeutic value. Folinic acid and its pharmaceutically acceptable salts, hereafter sometimes referred to as “active compounds”, are known and may be made in accordance with known procedures. See, generally The Merck Index, Monograph No. 4141 (11th Ed. 1989); U.S. Pat. No. 2,741,608.

The agent of the invention is provided in a pharmaceutical composition alone, or optionally in combination with other agents currently used to treat the diseases, conditions, symptoms and syndromes described above and other agents, some of which are listed below. The present product is of extreme value in subjects where existing treatments are either completely ineffective or partially effective at best or where, although the treatment may have been effective initially, its efficacy has eroded with time. The present method is effective in stimulating adenosine synthesis and, thereby, whether through direct adenosine action or through indirect action on other neural pathways and/or neurotransmitters, treat subjects afflicted with disorders or conditions who, for example, are associated with the use or administration of drugs or alcohol, with lack of deep sleep patterns, abnormally aggressive behavior, under- or overeating, prolonged periods of bed rest without exercising, mood disorders such as depression whether endogenous or as a consequence of physical conditions, such as impotence, surgery or traumatic body injury, and when afflicted by other diseases cancer, trauma, pain, surgery, viral infection, microbial infection, congestion, inflammation, auto-immune disease, menopause, sleeplessness, prolonged bed rest, surgery, bulimia, anorexia nervosa, wasting disorder, and genetic heritage and other conditions which result in, induce, or are associated with, mood abnormalities. Moreover, the present composition is effective for treating subjects who, whether as a consequence of trauma, surgery, the administration of an exogenous substance or any other cause, are afflicted with a disease or condition of the heart or vascular system, e.g. to treat or control the intensity and frequency of heart attacks, stroke, heart failure, heart fibrillation, SVT, ARDS, COPD, heart malfunction in general, for assessing heart function, and to treat other reduced adenosine level associated pathologies which result from other ailments, such as microbial infection, cancer, trauma, surgery, chronic pain, congestion, inflammation, auto-immune disease, and congenital disorders. The agent of the invention is provided in a pharmaceutical composition and may be used alone, or in combination with agents currently used to treat pain and inflammation associated with any disease, condition and syndrome. Examples of diseases, syndromes and conditions, whose associated pain and inflammation may be treated with the agent of this invention are arthritis, head aches, ear aches, head and peripheral body injuries, pre- and post-surgical pain, back pain, pain associated with child birth and pre- and post-partum pain, sport injuries, muscle pain associated with exercise and over exertion, pain associated with intake of steroids, osteoporosis, trauma, surgery, burns, wounds, chronic bronchitis, chronic obstructive pulmonary disease (COPD), allergic rhinitis, inflammatory bowel disease such as Crohn's disease and ulcerative colitis, autoimmune disease, and many others. Furthermore, folinic acid also has a sustainable activity for inducing, prolonging and deepening sleep patters and is, therefore, useful as a soporific (sleep inducing agent) and for the treatment and prevention of sleeplessness, restless sleep, cat-napping, insomnia, early awakening from sleep and low-wave sleep, among other sleep pathologies. He also found that folinic acid also is effective for inducing unconsciousness, sleep and anesthesia.

The term “adenosine depletion” is intended to encompass diseases and conditions such as heart attacks, stroke, heart failure, ischemia, fibrillation, SVT, ARDS, burns, trauma, surgery, mood disorders, pain and inflammation, lack of or restless sleep, and those that result from or in heart, lung and brain malfunction in general, which are associated with adenosine levels which are significantly reduced or depleted in one or more tissues, as compared to previous adenosine levels in the same subject or to a standard average level for the species (cut-off point), and conditions where adenosine levels are essentially the same as previous adenosine levels in that subject but, because of some other condition or alteration in that patient, a therapeutic benefit is achieved in the patient by increasing the adenosine levels as compared to previous levels. The present method is carried out, preferably, on patients where adenosine levels are reduced, e.g., by more than about 5%, about 10%, about 15%, about 20%, about 30% and more, to fully depleted as compared to previous adenosine levels in the subject. Although the present invention is primarily concerned with the treatment of human subjects, it also is employed for the treatment of vertebrates in general, particularly mammals. Among the animals treated may be domesticated and wild animals, large and small, for veterinarian purposes, and including house pets (cats, dogs and the like), zoo animals, race horses, farm animals (cows, sheep and the like) and many others.

In one embodiment, the pharmaceutical compositions provided herein comprise folinic acid and/or its salts as described above and one or more surfactants. Suitable surfactants or surfactant components for enhancing the uptake of the anti-sense oligonucleotides of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomeric and polymeric, poly (vinyl amine) with dextran and/or alkanoyl side chains, Brij 35, Triton X-100 and synthetic surfactants ALEC, Exosurf, Survan and Atovaquone, among others. These surfactants may be used either as single or part of a multiple component surfactant in a formulation, or as covalently bound additions to the 5′ and/or 3′ ends of the anti-sense oligonucleotides (oligos).

The second or additional agent of the present composition may be one or more of a variety of therapeutic and diagnostic agents which are suitable for administration to humans or non-human animals. Some of the categories of agents suitable for incorporation into the present composition and formulations are analgesics, pre-menstrual medications, anti-menopausal agents such as hormones, anti-aging agents, anti-anxiolytic agents, other mood altering agents, other anti-depressants, other anti-bipolar mood agents, other anti-schizophrenic agents, anti-cancer agents, alkaloids, heart medication, anti-anxiolytic agents, sleep inducing agents, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, etc.

Among the hormones are female and male sex hormones such as premarin, progesterone, androsterones and their analogues, thyroxine, glucocorticoids. Among the libido altering agents are Viagra, and NO-level modulating agents. Among the analgesics are over-the-counter medications such as ibuprofen, oruda, aleve, acetaminophen, and controlled substances such as morphine and codeine. Among the anti-depressants are tricyclics, MAO inhibitors and epinephrine, (-amino butyric acid (GABA), dopamine and serotonin level elevating agents such as Prozac, Amytryptilin, Wellbutrin and Zoloft. Among the skin renewal agents are Retin-A, hair growth agents such as Rogaine. Among the anti-inflammatory agents are non-steroidal anti-inflammatory drugs (NSAIDs) and steroids. Among the soporifics are melatonin and sleep inducing agents such as diazepam, cytoprotective, anti-ischemic and head injury agents such as enadoline, and many others. Examples of agents in the different groups are provided in the following list. Analgesics such as Acetaminophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti-anxiety agents associated with mental depression, such as Chlordiazepoxide, Amitriptyline, Loxapine Maprotiline and Perphenazine, among others. Anti-inflammatory agents such as non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Sedatives including Diphenhydramine, Hydroxyzine, Methotrimeprazine, Promethazine, Propofol, Melatonin, Trimeprazine, and the like. Sedatives and agents used for treatment of petit mat and tremors, among other conditions, such as Amitriptyline HCl; Chlordiazepoxide, Amobarbital; Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g. for treatment of severe head injury; orphan status, Warner Lambert), cytoprotective agents, and agents for the treatment of menopause, menopausal symptoms (treatment), e.g. Ergotamine, Belladonna Alkaloids and Phenobarbital, for the treatment of menopausal vasomotor symptoms, e.g. Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric treatments such as Tricyclic Antidepressants, including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications. Examples of anti-migraine agents are Imitrex and the like.

Pharmaceutically acceptable salts should be both pharmacologically and pharmaceutically acceptable. Such pharmacologically and pharmaceutically acceptable salts can be prepared as alkaline metal or alkaline earth salts, such as sodium, potassium or calcium salts, or the carboxylic acid group of folinic acid. The calcium salt of folinic acid is a preferred pharmaceutically acceptable salt. Organic salts and esters are also suitable for use with this invention. The active compounds are preferably administered to the subject as a pharmaceutical composition. Pharmaceutical compositions for use in the present invention include systemic and topical formulations, and among these preferred are formulations which are suitable for inhalation, oral, rectal, vaginal, nasal, ophthalmic, optical, intracavitary, intraorgan, topical (including buccal, sublingual, dermal and intraocular), parenteral (including subcutaneous, intradermal, intramuscular, intravenous and intraarticular) and transdermal administration, among others. The compositions may conveniently be presented in single or multiple unit dosage forms as well as in bulk, and may be prepared by any of the methods which are well known in the art of pharmacy. The composition of the invention may also be provided in the form of a kit, whether already formulated or with instructions for its formulation and administration regime. The kit may also contain other agents, such as those which were described above, and for example when for parenteral administration, also a carrier in a separate container, which may be sterile. The present composition may also be provided in a sterile contained for addition of a liquid carrier prior to administration. See, e.g. U.S. Pat. No. 4,956,355; UK Patent No. 2,240,472; EPO Patent Application Serial No. 429,187; PCT Patent Application Serial No. 91/04030, the relevant preparatory and compound portions of which are incorporated by reference above. See, also Mortensen, S. A., et al., Int. J. Tiss. Reac. XII(3): 155-162 (1990); Greenberg, S., et al., J. Clin. Pharm. 30: 596-608 (1990); Folkers, K., et al., Proc. Nat'l. Acad. Sci. 87: 8931-8934 (1990), the relevant preparatory and compounding portions of which are also incorporated herein by reference. Formulations suitable for oral and parenteral administration are preferred, and inhalable preparations are even more preferred. All methods include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and a suitable carrier. In general, the compositions of the invention are prepared by uniformly and intimately admixing the active compound with a liquid or finely divided solid carrier, or both, and then, if necessary, shaping the resulting mixture. For example, a tablet may be prepared by compressing or molding a power or granules containing the active compound, optionally with one or more accessory ingredients. Compressed tablets may be prepared by compressing, in a suitable machine, the compound in a free-lowing form, such as a powder or granules optionally mixed with a binder, lubricant, inert diluent, and/or surface active/dispensing agent (s). Molded tablets may be made by molding, in a suitable machine, the powdered compound moistened with an inert liquid binder. Compositions for oral administration may optionally include enteric coatings known in the art to prevent degradation of the compositions in the stomach and provide release of the drug in the small intestine. Compositions suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Compositions suitable for parenteral administration comprise sterile aqueous and non-aqueous injection solutions of the active compound, which preparations are preferably isotonic with the blood of the intended recipient. These preparations may contain antioxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Compositions suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof. Compositions suitable for transdermal administration may be presented as discrete patches adapted to remain in intimate contact with the epidermis of the recipient for a prolonged period of time. Compositions suitable for transdermal administration may also be delivered by iontophoresis, and typically take the form of an optionally buffered aqueous solution of the active compound. See, e.g. Pharmaceutical Research 3: 318 (1986).

The active compound of this invention is provided within broad amounts of the composition. For example, folinic acid, its salts and their mixtures may be contained in the composition in amounts of about 0.001%, about 0.1%, about 1%, about 2%, about 5% to about 99.999%, about 98%, about 90%, about 40%, about 20%, about 10%, about 5% of the composition, preferably about 1 to about 99%, more preferably about 2% to 40%, and still more preferably about 2% to about 10% of the composition. These amounts may be adjusted when and if additional agents with overlapping activities are included as discussed above.

The dosage of the active compound may vary depending on age, weight, and condition of the subject. Treatment may be initiated with a small dosage which is less than the optimal dose of the first agent of the invention, be it folinic acid or one of its salts. The dose may be increased until a desired and/or optimal effect under the circumstances is reached. In general, the dosage is about 1, about 5, about 10, about 20, about 50 mg/kg body weight and up to about 100, about 200, about 500 or about 1000 mg/kg body weight. Currently, preferred are dosages of about 5 to about 500 mg/kg body weight of the subject, more preferred are dosages of about 10 to about 200 mg/kg, and still more preferred are dosages of about 50 to about 100 mg/kg body weight of the subject. Higher or lower doses, however, are also contemplated and are, therefore, within the confines of this patent. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, i.e. folinic acid and its salts, the dose of one of the other or of both agents may be adjusted to attain a desirable effect without exceeding a dose range which avoids untoward side effects. Thus, for example, when other analgesic and anti-inflammatory agents are added to the composition, they may be added in amounts known in the art for their intended application or in doses somewhat lower that when administered by themselves.

In general, the present composition is provided in a variety of systemic and topical formulations. The systemic or topical formulations of the invention are selected from the group consisting of oral, intrabuccal, intrapulmonary, rectal, intrauterine, intradermal, topical, dermal, parenteral, intratumor, intracranial, buccal, sublingual, nasal, intramuscular, subcutaneous, intravascular, intrathecal, inhalable, transdermal, intraarticular, intracavitary, implantable, transdermal, iontophoretic, intraocular, ophthalmic, vaginal, intraarticular, otical, intravenous, intramuscular, intraglandular, intraorgan, intralymphatic, implantable, slow release and enteric coating formulations. The actual preparation and compounding of these different formulations is known in the art and need not be detailed here. The active compounds may be administered once or several times a day. The active compounds disclosed herein may be administered to the lungs of a subject by any suitable means, but are preferably administered by generating an aerosol comprised of respirable particles, the respirable particles comprised of the active compound, which particles the subject inhales, i.e. by inhalation administration. The respirable particles may be liquid or solid. Particles comprised of active compound for practicing the present invention should include particles of respirable size: that is, particles of a size sufficiently small to pass through the mouth and larynx upon inhalation and into the bronchi and alveoli of the lungs. In general, particles ranging from about 0.5 to about 10 microns in size, more particularly, less than about 5 microns in size, are respirable. Particles of non-respirable size which are included in the aerosol tend to deposit in the throat and be swallowed, and the quantity of non-respirable particles in the aerosol is preferably minimized. For nasal administration, a particle size in the range of 10-500 μm is preferred to ensure retention in the nasal cavity.

Liquid pharmaceutical compositions of the active compound suitable for producing an aerosol may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optional contain a dispersant which serves to facilitate the formation of an aerosol. A suitable dispersant is lactose, which may be blended with the active compound in any suitable ratio, e.g. a 1 to 1 ratio by weight.

Aerosols of liquid particles comprising the active compound may be produced by any suitable means, such as with a nebulizer. See, e.g. U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w of the compositions, but preferably less than 20% w/w of the carrier is typically water or a dilute aqueous alcoholic solution, preferably made isotonic with body fluids by the addition of, for example sodium chloride. Optional additives include preservatives if the compositions are not prepared sterile, for example, methyl hydroxybenzoate, antioxidants, flavoring agents, volatile oils, buffering agents and surfactants.

Aerosols of solid particles comprising the active compound may likewise be produced with any sold particulate medicament aerosol generator. Aerosol generators for administering solid particulate medicaments to a subject produce particles which are respirable, as explained above, and they generate a volume of aerosol containing a predetermined metered dose of a medicament at a rate suitable for human administration. Examples of such aerosol generators include metered dose inhalers and insufflators.

The second agent(s) may be administered concurrently with the active compounds for preventing and treating mood disorders, preferably in the same composition, as described above. The phrase “concurrent administration”, as used herein, means that the folinic acid or its salt and the second agent(s) are administered either (a) simultaneously in time, and preferably by formulating the two together in a common pharmaceutical carrier, or (b) at different times during the course of a common treatment schedule. In the latter case, the two compounds are administered at times effective to complement their half lives and, thereby offset a reduction in peak level of one with an increasing level of the other and, thereby, counter balance any decrease in activity of one with an increase in activity of the other as a result of their alternate administration schedule. Thus the active compound may or may not be administered for a time sufficient to bring endogenous adenosine levels back to prior levels in the subject. If the present composition or formulations are administered for a time sufficient to replenish endogenous adenosine levels (if lowered with respect to prior levels in the same subject), then the folinic acid, its salts or their mixtures and the second agent are administered in amounts effective to increase adenosine levels to a desired level. Thereafter, the doses of the two or more agents may be reduced so as to maintain adenosine levels, whether the second agent has overlapping activity with the active compound or, if of different activity, the dose of the second agent may be reduced along with that of the active compound in cases of reduced risk of relapse. If the active compound is administered for a time sufficient to replenish endogenous adenosine levels, and this is attained, the continuation of treatment will depend on whether the adenosine levels are maintained in the absence of treatment or not. Moreover, whether the dose of the second agent(s) is reduced or not will depend on whether or not it is necessary to continue its administration or the subject remains stable. If the practitioner perceives a need to offset a future relapse, be it as a decrease in adenosine levels or even its depletion and/or a need or benefit from a continued administration of the second agent (s), the treatment may be continued with close monitoring.

The additional agents, examples of which are listed above, may be administered per se or in the form of pharmaceutically acceptable salts. When used in medicine, the salts of these agents should be pharmacologically and pharmaceutically acceptable, but non-pharmaceutically acceptable salts may be used as well to prepare the free active compound or pharmaceutically acceptable salts thereof and are not excluded from the scope of this invention. Such pharmacologically and pharmaceutically acceptable salts include, but are not limited to, those prepared from the hydrochloric, hydrobromic, sulfuric, nitric, phosphoric, maleic, acetic, salicylic, p-toluenesulfonic, tartaric, citric, methanesulphonie, formic, malonic, succinic, naphthalene-2-sulphonic and benzenesulphonic acids, among others. Pharmaceutically acceptable salts also may be prepared as alkaline metal or alkaline earth salts, such as sodium, potassium or calcium salts of the carboxylic acid group. The present pharmaceutical formulations, whether veterinary or human use, may comprise, in addition to the active compound and one or more additional agents, one or more pharmaceutically acceptable carriers, and optionally any other therapeutic ingredients suitable for specific types of diseases and conditions. The carrier(s) must be pharmaceutically acceptable in the sense of being compatible with the other ingredients of the formulation and not unduly deleterious to the recipient thereof

Formulations of the present composition suitable for oral administration may be presented as discrete units such as capsules, cachets, tablets or lozenges, each containing a predetermined amount of the potentiating agent as a powder or granules; or a suspension in an aqueous liquor or nonaqueous liquid such as a syrup, an elixir, an emulsion or a draught.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised of a mixture of the powdered active compound with a suitable carrier may be made by molding in a suitable machine.

A syrup may be made by adding the active compound to a concentrated aqueous solution of a sugar, for example sucrose to which may also be added any accessory ingredient(s). Such accessory ingredient(s) may include flavorings, suitable preservatives, an agent to retard crystallization of the sugar, and an agent to increase the solubility of any other ingredient, such as a polyhydric alcohol, for example glycerol or sorbitol.

Formulations suitable for parenteral administration conveniently comprise a sterile aqueous preparation of the active compound, which is preferably isotonic with the blood of the recipient.

Nasal spray formulations comprise purified aqueous solutions of the active compound with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye. Otical formulations are generally prepared in viscous carriers, such as oils and the like, as is known in the art, so that they may be easily administered into the ear without spilling.

Topical formulations comprise the active compound dissolved or suspended in one or more media such as mineral oil, petroleum, polyhydroxy alcohols or other bases used for topical pharmaceutical formulations. The addition of other accessory ingredients, vide infra, may be desirable.

In addition to the aforementioned ingredients, the formulations of this invention may further include one or more accessory ingredient(s) selected from diluents, buffers, surfactants, propellants, flavoring agents, binders, disintegrants, surface active agents, thickeners, lubricants, preservatives (including antioxidants) and the like. Other ingredients may also be utilized as is known in the art.

The agent of this invention may be administered in combination with other treatments, chemical or not, and including psychotherapy, particularly treatments that act by independent mechanisms, to thereby provide a multi-pronged attack on the mood disorder. Thus, the agent of the invention may be used alone, with a carrier, with other treatments or agents, as a foodstuff additive, or in other compositions suitable for human consumption. This invention, thus, also provides an edible mood regulating product, which comprises a foodstuff or its ingredients; and a mood regulating effective amount of the first agent. Each one of these agents may be used alone or combined with one or more of the second agents provided herein, or further combined with a foodstuff or food supplement for self-administration. The agent of this invention may also be provided in a kit, in separate containers, with other agents including, but not restricted to, second agent(s), vitamin supplements, mineral additives, other nutritional additives, buffers, salts, flavoring compounds, diluents, thickeners, emulsifiers, preservatives, and anti-oxidants, such as would be familiar to a person skilled in the art, as would the amounts they are added in to the composition. The composition or product may also comprise a binder such as gum tragacanth, acacia, corn starch or gelatin, excipients such as dicalcium phosphate, anti-clumping agents such as corn starch, potato starch, alginic acid and the like, lubricants such as magnesium stearate, sweetening agents such as sucrose, lactose or saccharin, flavoring agents such as peppermint, orange, wintergreen or cherry flavoring as well as other known artificial and natural flavoring compounds. Sustained-release preparations and formulations are also within the confines of this invention, and may contain further ingredients as is know in the art.

A coated composition, or otherwise modified forms of the preparation are also contemplated herein such as coatings of shellac, gelatin, sugar and the like. Any material added to this product should be pharmaceutically-acceptable and substantially non-toxic in the amounts employed. Other excipients may be added to the formulation such as those utilized for the production of ingestible tablets, troches, capsules, elixirs, suspensions, syrups and wafers, among others and the product may then be provided in these forms. The agent of this invention may be present in the edible product in an amount of about 0.01 to 99.99 wt % of the product, and preferably about 0.1 to 20.0 wt %. Other amounts of the agent, however, may also be present in the edible product. The amount of the agent in the pharmaceutical composition and in the edible product may be varied, and/or the frequency of administration increased or decreased, depending on the severity of the subject's condition, the general health and nutritional status of the subject, and whether or not other mood regulating agents are being administered as well.

Foodstuffs suitable for use in the mood controlling product of the invention are milk, juices, cereals, chewing gum, crackers, candies, meats, vegetables and fruits, blended or otherwise as baby food for example, and cookies, among others. The present agent, however, may also be added to salads, meat, fish and poultry dishes, as well as to deserts, either when these edible products are being prepared or at the table.

The agent of this invention is also provided as a mood regulating kit, which comprises a mood regulating, heart, brain, lung and vasculature protecting, analgesic, anti-inflammatory, soporific, anesthetic, and heart function testing composition comprising the first agent, and optionally other therapeutic agent(s), carrier(s) and foodstuff ingredients; and instructions use of the kit for admixing the composition with other ingredients in preparing an edible product. The kit may also be provided with other baking or cooking ingredients needed for particular recipes, such as cakes, pudding, cookies, sauces, drinks, and the like. In a particularly preferred embodiment, the kit has each and every ingredient sealed wrapped in separate containers, and has one or more recipes for preparation of specific products. This kit is formulated for the therapeutic treatment of subjects afflicted with or at risk of mood disorders associated with adenosine levels. Other ingredients may be added to the edible product, including vitamin supplements, mineral additives, other nutritional additives, salts, buffers, flavoring compounds, diluents, thickeners, emulsifiers, surfactants, preservatives, and anti-oxidants, such as would be familiar to a person skilled in the art. Included within the invention, is an embodiment wherein the above compositions further comprise varying amounts of other components such as foodstuffs. Suitable are all kinds of foods including milk and milk supplements. The composition or the edible product of the invention may also be modified to include varying amounts of water and ingredients suitable to the clinical needs of the subject.

The composition may be mixed with a drink (liquid) or a foodstuff for self-administration. The composition may be added in a mood disorder controlling, heart, brain, lung or vasculature protecting, analgesic, anti-inflammatory, soporific, sleep inducing, anesthetic, etc., effective amount, and may be provided in bulk or in unit form. Embodiments which are particularly suitable for use while traveling are in the form of chewing gum, candy (hard and soft), and as a powder for addition to a drink prior to ingestion.

The agent of this invention exhibits an additional advantage for the treatment of the types of conditions described because its components are endogenous to the human body. The present agent is thus unlikely to elicit toxic, immunological or allergic reactions in treated subjects. Because this agent is innocuous to the human body, the invention may be used without intervention of skilled medical personnel, for example, by adding it to foodstuffs, and the like, that are normally sold over-the-counter in convenience stores or as food supplements available in grocery stores. This is a particular advantage for treating travelers or populations in underdeveloped countries where medical services are in short supply.

Having now generally described this invention, the same will be better understood by reference to certain specific examples, which are included herein for purposes of illustration only and are not intended to be limiting of the invention or any embodiment thereof, unless so specified.

### EXAMPLES

In the following examples, and throughout this patent, “DHEA” means dehydroepiandrosterone, “F.A.” means folinic acid, “M” means methyltestosterone, “s” means seconds, “mg” means milligrams, “kg” means kilograms, “kw” means kilowatts, “Mhz” means megahertz, and “nmol” means nanomoles.

### Examples 1 and 2

### In Vivo Effects of Folinic Acid & DHEA on Adenosine Levels

Young adult male Fischer 344 rats (120 grams) were administered dehydroepiandrosterone (DHEA) (300 mg/kg) or methyltestosterone (40 mg/kg) in carboxymethylcellulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen with 10 seconds of death. Liver and lungs were removed en bloc and flash frozen with 30 seconds of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 2 below. Results are expressed as the mean “SEM, with X p<0.05 compared to control group and i p<0.05 compared to DHEA or methyltestosterone-treated groups.

TABLE 2
In vivo Effect of DHEA, *-1-methyltestosterone &
Folinic Acid an Adenosine Levels in various Rat Tissues
Intracellular Adenosine
(nmol/mg protein)

| | | | |
|---|---|---|---|
| Treatment | Heart | Lung | Brain |
| Control | 10.6 ± 0.6 | 3.1 ± 0.2 | 0.5 ± 0.04 |
| | (n = 12) | (n = 6) | (n = 12) |
| DHEA | 6.7 ± 0.5 | 2.3 ± 0.3 | 0.19 ± 0.01 |
| (300 mg/kg) | (n = 12) | (n = 6) | (n = 12) |
| Methyltestost. (M) | 8.3 ± 1.0 | N.D. | 0.42 ± 0.06 |
| (40 mg/kg) | (n = 6) | | (n = 6) |
| Methyltestost. (M) | 6.0 ± 0.4 | N.D. | 0.32 ± 0.03 |
| (120 mg/kg) | (n '2 6) | | (n = 6) |
| Follinic Acid (F.A.) | 12.4 ± 2.1 | N.D. | 0.72 ± 0.09 |
| (50 mg/kg) | (n = 5) | | (n = 5) |
| M + F.A. | 9.1 ± 0.4 | N.D. | 0.60 ± 0.06 |
| (120 mg/kg/kg; 50 mg/ | (n = 6) | | (n = 6) |
| kg) | | | |

N.D. = Not Determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Co-administration of folinic acid completely abrogated adenosine depletion. Folinic acid administered alone induce increase in adenosine levels for all organs studied.

The invention now being fully described, it will be apparent to one of ordinary skill in the art that many changes and modifications can be made thereto without departing from the spirit or scope of the invention as set forth therein.

95. A pharmaceutical composition, comprising a pharmaceutically or veterinarily acceptable carrier and an amount of a first agent effective as an anti-pyretic, analgesic, unconsciousness inducer or anesthetic, or as an aggressive behavior or neutrophil function regulator, or as a protector or inducer of reperfusion of ischemic or stroke afflicted organs, tissue, cell or vasculature, or for alleviating chronic bronchitis, COPD, allergic rhinitis, auto-immune diseases, wounds, burns, inflammation, sequelae of menopause, bed sores or heart conditions, or for assessing heart or cardiovascular function, the first agent being selected from folinic acid, its physiologically acceptable salts or mixtures thereof.

96. The composition of claim 95, further comprising a second agent selected from analgesics, anti-pre-menstrual syndrome (PMS) agents, anti-menopausal agents, anti-aging agents, anti-anxiolytic agents, mood controlling agents, anti-depressants, anti-bipolar mood disorder agents, anti-schizophrenic agents, anti-cancer agents, aggression controlling agents, anti-attention deficit disorder agents, anti-delirium agents, anti-compulsive obsessive disorder (COD) agents, anti-seizure agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents or hair growth agents.

97. The composition of claim 95, wherein the first agent is provided in an amount effective for stabilizing or regulating heart activity, for alleviating or controlling pain or inflammation, for preventing or treating viral or microbial infection, skin lacerations, organ or tissue trauma, burns, restless sleep, stroke, heart failure, arrythmias, heart fibrillation, SVT, RDS, chronic bronchitis, COPD, allergic rhinitis or auto-immune disease or intensity, frequency or severity of heart attacks or strokes, or for inducing unconsciousness, α-wave sleep or anesthesia.

98. The composition of claim 97, wherein the pain or inflammation is associated with bed sores, burns, trauma, Crohn's disease, ulcerative colitis (UC), surgery or arthritis.

99. The composition of claim 97, wherein the pain or inflammation is associated with head aches, ear aches, head or peripheral body injury, pre- and post-surgical pain, back pain, child birth pain, pre- and post-partum pain, sport injuries, exercise or over-exertion pain, intake of steroids, osteoporosis, trauma, surgery, burns, wounds, sores, chronic bronchitis, chronic obstructive pulmonary disease (COPD), allergic rhinitis, inflammatory bowel disease (IBD) or autoimmune disease.

100. The composition of claim 95, further comprising a surfactant selected from surfactant protein A, surfactant protein B, surfactant protein C, surfactant protein D and surfactant protein fragments thereof, non-dipalmitoyl disaturated phosphatidylcholine, dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholin, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate, artificial lamellar bodies vehicles for surfactant components, omega-3 fatty acids, polyenic acid, polyenoic acid, lecithin, palmitic acid, non-ionic ethylene and/or propylene oxide block copolymers, polyoxypropylene, polyoxyethylene, poly (vinyl amine) with dextran and/or alkanoyl side chains, Brij 35®, Triton X-100®, ALEC®, Exosurf®, Survant® or Atovaquone®.

100. The composition of claim 95, wherein the carrier comprises a liquid or solid carrier; and the composition comprise4s a systemic or topical formulation.

101. The formulation of claim 95, further comprising anti-oxidants, flavoring agents, volatile oils, buffering agents, dispersants, propellants, preservatives or surfactants.

102. The formulation of claim 100, wherein the formulation is selected from oral, intrabuccal, intrapulmonary, rectal, intrauterine, intradermal, topical, dermal, parenteral, intratumor, intracranial, buccal, sublingual, nasal, intramuscular, subcutaneous, intravascular, intrathecal, inhalable, transdermal, intraarticular, intracavitary, implantable, transdermal, iontophoretic, intraocular, ophthalmic, vaginal, intraarticular, otical, intravenous, intramuscular, intraglandular, intraorgan, intralymphatic, implantable, slow or sustained release or enteric coating formulations.

103. The composition of claim 102, which is an oral formulation selected from the group consisting of capsules, cachets, lozenges, tablets, powder, granules, solutions, suspensions and emulsions.

104. The oral formulation of claim 103, wherein the solutions or suspensions comprise aqueous or non-aqueous liquid solutions or suspensions, and the emulsions comprise oil-in-water or water-in-oil emulsions.

105. The oral formulation of claim 102, which is a buccal or sub-lingual formulation selected from lozenges that may further comprise a flavoring agent selected from sucrose, acacia or tragacanth; or pastilles that may further comprise an inert base selected from gelatin, glycerin, sucrose or acacia.

106. The oral formulation of claim 103, further comprising an enteric coating.

107. The composition of claim 102, which is a parenteral formulation selected from injectable solutions, suspensions or emulsions that may further comprise anti-oxidants, buffers, bacteriostatic agents or solutes which render the solution or suspension isotonic with blood.

108. The parenteral formulation of claim 107, wherein the solutions, suspensions or emulsions are selected from sterile aqueous or non-aqueous injection solutions, suspensions or emulsion that may further comprise suspending agents or thickening agents.

109. A sterile ampoule or vial, comprising the parenteral formulation of claim 107.

110. The composition of claim 95, which is in bulk, or in single- or multi-unit dose form.

111. The composition of claim 95, which is freeze-dried or lyophilized.

112. The composition of claim 103, which is a topical formulation selected from ointments, creams, lotions, pastes, gels, sprays, aerosols or oils.

114. The topical formulation of claim 112, wherein the carrier is selected from vaseline, lanoline, polyethylene glycols, alcohols or transdermal transport enhancers.

115. The composition of claim 112, which is a transdermal formulation.

116. The transdermal formulation of claim 115, comprising a solution, suspension or emulsion of the first agent, which may further comprise a buffer and one or more second agent(s).

117. A transdermal delivery device, for delivery of the formulation of claim 115.

118. A transdermal delivery device, comprising the formulation of claim 115, which may comprise a patch.

119. The formulation of claim 103, which is an inhalable, nasal, respirable or intrapulmonary formulation.

120. The formulation of claim 119, which is an aerosol comprising liquid or solid particles that may further comprise an agent selected from preservatives, antioxidants, flavoring agents, volatile oils, buffering agents, dispersants or surfactants.

121. The formulation of claim 119, comprising a powder or liquid formulation of respirable, nasal, intrapulmonary or inhalable particles about 0.5 to about 10 μm in size or about 10 to about 500 μm in size.

122. The formulation of claim 121, comprising particles about 0.5 up to about 5 μm in size.

123. The formulation of claim 120, which is an aerosol powder comprising solid particles about 0.5 to about 10 μm in size or about 10 to about 500 μm in size.

124. The formulation of claim 120, which is a liquid aerosol of particles about 0.5 to about 10 μm in size or about 10 to about 500 μm in size.

125. The formulation of claim 119, provided in a capsule(s) or cartridge(s).

126. The composition of claim 102, wherein the carrier comprises a hydrophobic carrier.

127. A delivery device, for nasal, inhalation, respiratory or intrapulmonary delivery of the formulation of claim 119.

128. A delivery device for nasal, respiratory, inhalation or intrapulmonary delivery, comprising the formulation of claim 119.

129. The device of claim 127, comprising an inhalator.

130. The device of claim 129, wherein the inhalator comprises a nebulizer or insufflator.

131. The delivery device of claim 129, comprising a pressurized inhalator.

132. The delivery device of claim 127, comprising a device that delivers a pre-measured dose(es) of the formulation.

133. A kit comprising a delivery device for nasal, inhalation, respiratory or intrapulmonary delivery device, in a separate container the agent of claim 95, and instructions for preparation of a nasal, inhalation, respiratory or intrapulmonary formulation and for its delivery.

134. The kit of claim 133, wherein the device comprises an inhalator.

135. The kit of claim 134, wherein the inhalator comprises a nebulizer or insufflator that may receive, and be operated to pierce or open, a capsule or cartridge, and the kit further comprises a piercable or openable capsule or cartridge with solid or liquid particles of the composition.

136. The kit of claim 133, wherein the delivery device comprises a pressurized inhalator, and the instructions provided are for the preparation of a formulation comprising a suspension or solution in an aqueous or non-aqueous liquid or an oil-in-water or water-in-oil emulsion.

137. A kit for nasal, inhalation, respiratory or intrapulmonary delivery, comprising a device for nasal, inhalation, respiratory or intrapulmonary delivery, in a separate container the formulation of claim 119, and instructions for its use.

138. The kit of claim 133, further comprising in a separate container a surfactant.

139. The kit of claim 138, wherein the surfactant is selected from surfactant protein A, surfactant protein B, surfactant protein C, surfactant protein D and surfactant protein and active fragments thereof, non-dipalmitoyl disaturated phosphatidylcholine, dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholin, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate, artificial lamellar bodies vehicles for surfactant components, omega-3 fatty acids, polyenic acid, polyenoic acid, lecithin, palmitic acid, non-ionic ethylene and/or propylene oxide block copolymers, polyoxypropylene, polyoxyethylene, poly (vinyl amine) with dextran and/or alkanoyl side chains, Brij 35, Triton X-100, ALEC, Exosurf, Survant or Atovaquone.

140. The composition of claim 103, which comprises a suppository(ies).

141. The composition of claim 103, which comprises an implant(s).

142. The composition of claim 102, which comprises a slow or sustained release formulation.

143. The composition of claim 103, which comprises an ophthalmic formulation.

144. The composition of claim 103, which comprises an otical formulation.

145. The composition of claim 103, which comprises a vaginal formulation selected from creams, gels, suppositories or implants.

146. The composition of claim 96, comprising a carrier, a first agent(s), and a second agent(s) selected from analgesic agents, anti-inflammatory agents, muscle relaxant agents, sleep inducing agents, anti-anxiolytic agents, diuretics, mood altering agents, vitamins, minerals, proteins, or hormonal agents.

147. The composition of claim 146, wherein the second agent(s) comprise(s) a mood regulating agent(s).

148. The composition of claim 146, wherein the second agent(s) comprise(s) a diuretic(s).

149. The composition of claim 146, wherein the second agent(s) comprise(s) vitamins and/or minerals.

150. The composition of claim 146, wherein the second agent(s) comprise(s) an anti-anxiolytic agent(s).

151. The composition of claim 146, wherein the second agent(s) comprise(s) one or more analgesic and/or anti-inflammatory agent(s).

152. The composition of claim 146, wherein the second agent(s) comprise(s) one or more hormonal agent(s).

153. The composition of claim 146, comprising the first agent(s), a hormone(s) having estrogen and progesterone activity, a diuretic(s), an analgesic(s) or an anti-inflammatory agent(s).

154. The composition of claim 115, which comprises an iontophoretic transdermal formulation further comprising an iontophoretic transport promoting agent(s), wherein the carrier is selected from solutions, suspensions or emulsions that comprise an aqueous or alcoholic solution or suspension, an oily solution or suspension or an oil-in-water or water-in-oil emulsion.

155. An iontophoretic delivery device, for delivery of the formulation of claim 154.

156. An iontophoretic device, comprising the formulation of claim 154.

157. An implantable capsule or cartridge, comprising the transdermal formulation of claim 115.

158. A transdermal delivery kit comprising, in separate containers,
a transdermal delivery device;
the agent of the formulation of claim 115; and
instructions for preparing the formulation and for its use.

159. The formulation of claim 126, wherein the hydrophobic carrier is selected from lipid vesicles or particles.

160. The formulation of claim 159, wherein the vesicles comprise liposomes, or the particles comprise microcrystals.

161. The composition of claim 160, wherein the liposomes comprise the first agent, and optionally one or more second agent(s) selected from analgesics, anti-pre-menstrual syndrome (PMS) agents, anti-menopausal agents, anti-aging agents, anti-anxiolytic agents, mood controlling agents, anti-depressants, anti-bipolar mood disorder agents, anti-schizophrenic agents, anti-cancer agents, aggression controlling agents, anti-attention deficit disorder agents, anti-delirium agents, anti-compulsive obsessive disorder (COD) agents, anti-seizure agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents; skin renewal agents or hair growth agents.

162. A method for inducing anti-pyresis, unconsciousness or anesthesia, or for regulating neutrophil function, prophylactically or therapeutically protecting or inducing reperfusion of ischemic or stroke afflicted endogenous and in-transplantation organs, tissues, cells or vasculature, or for alleviating chronic bronchitis, COPD, allergic rhinitis, auto-immune disease, bed sores, wounds, burns, inflammation, sequelae of menopause, heart conditions, or for assessing heart or cardiovascular function, comprising administering to a subject in need of treatment the pharmaceutical composition of claim 95, comprising an anti-pyretic, analgesic, unconsciousness inducing or anesthetic, an aggressive behavior or neutrophil function regulating, a reperfusion of ischemic or stroke afflicted organ, tissue, cell or vasculature protective or inducing, a chronic bronchitis, COPD, allergic rhinitis, auto-immune diseases, wound, burn, inflammation, sequelae of menopause, bed sores, or heart condition alleviating, or a heart or cardiovascular function assessing effective amount of the first agent of claim 95.

163. The method of claim 162, wherein the first agent is administered in an amount of about 1 to about 1,000 mg/kg body weight.

164. The method of claim 162, for stabilizing or regulating heart activity, for preventing or treating inflammation, sores, burns, trauma, Crohn's disease, ulcerative colitis (UC), surgery, arthritis, viral or microbial infection, skin lacerations, organ or tissue trauma, burns, restless sleep; stroke, heart failure, arrythmias, heart fibrillation, SVT, RDS, chronic bronchitis, COPD, allergic rhinitis, the intensity, frequency or severity of heart attacks or stroke or auto-immune disease, or for inducing unconsciousness, α-wave sleep or anesthesia.

165. The method of claim 162, for preventing or treating arthritis, head aches, ear aches, body injury, pre- and post-surgical pain, back pain, pain associated with child birth, pre- and postpartum pain, sport injuries, muscle pain associated with exercise or over-exertion, pain associated with intake of steroids, osteoporosis, trauma, surgery, burns, wounds, chronic bronchitis, chronic obstructive pulmonary disease (COPI)), allergic rhinitis, inflammatory bowel disease (IBD) or autoimmune disease.

166. The method of claim 162, further comprising administering to the subject a second agent selected from analgesics, anti-pre-menstrual syndrome agents, anti-menopausal agents, anti-aging agents, anti-anxiolytic agents, mood controlling agents, anti-depressants, anti-bipolar disorder agents, anti-schizophrenic agents, anti-cancer agents, alkaloids, blood pressure controlling agents, hormonal agents, anti-inflammatory agents, muscle relaxants, steroids, soporific agents, anti-ischemic agents, anti-arrhythmic agents, contraceptives, vitamins, minerals, tranquilizers, soporifics, appetite suppressants, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart medicines, adenosine, adenosine releasing agents, adenosine metabolism inhibitors, or adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents or hair growth agents.

167. The method of claim 166, wherein the second agent comprises an analgesic, anti-inflammatory, mood regulating agent, muscle relaxant or sleep regulating agent(s).

168. The method of claim 162, wherein the composition is administered systemically or topically.

169. The method of claim 168, wherein the composition is administered buccally, sublingually, dermally, intraocularly, subcutaneously, intradermally, intramuscularly, intravenously, intraarticularly, intrapulmonarily, rectally, intrauterinely, parenterally, intratumorly, intracranially, nasally, intravascularly, intrathecally, transdermally, intracavitarily, iontophoretically, ophthalmic, vaginally, otically, intraglandularly, intraorgan or lymphatically, or by inhalation or implantation, or into the respiration.

170. The method of claim 169, wherein the composition is administered orally as a formulation selected from capsules, cachets, lozenges, tablets, powder, granules, solutions, suspensions or emulsions.

171. The method of claim 170, wherein the oral formulation further comprises an enteric coating.

172. The method of claim 169, wherein the composition is administered buccally or sub-lingually in the form of lozenges that may further comprise a flavoring agent(s) selected from sucrose, acacia or tragacanth; or pastilles that may further comprise an inert base selected from gelatin, glycerin, sucrose or acacia.

173. The method of claim 169, wherein the composition is administered parenterally in the form of an injectable solution, suspension or emulsion, which may further comprise antioxidants, buffers, bacteriostatic agents, or solutes that render the solution, suspension or emulsion isotonic with blood.

174. The method of claim 162, wherein the composition is freeze-dried or lyophilized; and the method further comprises adding a sterile liquid or solid carrier prior to use.

175. The method of claim 169, wherein the composition is administered topically as an ointment, cream, lotion, paste, gel, spray, aerosol or oil that may further comprise vaseline, lanoline, polyethylene glycols, alcohol or trans-dermal transport enhancers.

176. The method of claim 169, wherein the composition is administered transdermally from a patch.

177. The method of claim 162, further comprising administering a surfactant selected from surfactant protein A, surfactant protein B, surfactant protein C, surfactant protein D and surfactant protein fragments thereof, non-dipalmitoyl disaturated phosphatidylcholine, dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholin, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate, artificial lamellar bodies vehicles for surfactant components, omega-3 fatty acids, polyenic acid, polyenoic acid, lecithin, palmitic acid, non-ionic ethylene and/or propylene oxide block copolymers, polyoxypropylene, polyoxyethylene, poly (vinyl amine) with dextran and/or alkanoyl side chains, Brij 35®, Triton X-100®, ALEC®, Exosurf®, Survant® or Atovaquone®.

178. The method of claim 177, wherein the first agent and the surfactant are administered in one composition.

179. The method of claim 169, wherein the composition is administered by inhalation, respiration, nasaly or intrapulmonarily.

180. The method of claim 179, wherein the composition is administered as an inhalable, respirable, nasal or intrapulmonary formulation comprising an aerosol of liquid solid powdered particles.

181. The method of claim 162, wherein the composition further comprises preservatives, antioxidants, flavoring agents, volatile oils, buffering agents, dispersants or surfactants.

182. The method of claim 180, wherein the aerosol comprises particles about 0.5 to about 10 μm in size or about 10 to about 500 μm in size.

183. The method of claim 180, wherein the aerosol comprises particles about 0.5 to about 5 μm in size.

184. The method of claim 169, wherein the composition is administered vaginally or rectally.

185. The method of claim 169, wherein the composition is administered ophthalmically or otically.

186. The method of claim 169, wherein the composition is administered intraarticularly.

187. The method of claim 162, wherein the composition further comprises other therapeutic agent(s), a preservative(s), an anti-oxidant(S), a flavoring agent(s), a volatile oil(s), a buffering agent(s), a dispersant(s) or surfactant(s).

188. The method of claim 162, wherein the subject is a human or a non-human animal.

189. The method of claim 162, for prophylaxis or treatment of a disease or condition associated with burns, trauma, inflammation, pain, surgery, viral infection, microbial infection, ischemia, congestion, inflammation, auto-immune disease, menopause, prolonged bed rest, surgery, bulimia, anorexia nervosa, or wasting disorder.

190. The method of claim 189, wherein the disease or condition is also associated with sleeplessness, cancer or a mood disorder.

191. The method of claim 190, wherein the patient is prone to, or is afflicted with, depression, bipolar disease, schizophrenia, over-aggressiveness, attention deficit disorder (ADD), delusions, compulsive obsessive disorder (COD) or delirium.

192. The method of claim 189, wherein the patient is subject to an operation, traumatic body injury, chronic pain or cancer.

193. The method of claim 160, wherein the first agent is administered in an amount of about 1 to about 1,000 mg/kg body weight of the subject.

194. The method of claim 162, further comprising administering to the subject a second agent selected from analgesics, anti-pre-menstrual syndrome agents, anti-menopausal agents, anti-aging agents, anti-anxiolytic agents, other mood disorder regulating agents, anti-depressants, anti-bipolar mood disorder agents, anti-schizophrenic agents, anti-attention deficit disorder (ADD) agents, anti-cancer agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, hormonal agents, anti-arthritic agents, anti-compulsive obsessive disorder (COD) agents, aggression level lowering agents, anti-angiogenic agents, cytokines, growth factors, anti-metastatic agents, antacids, anti-histaminic agents, anti-bacterial agents, anti-viral agents, anti-gas agents, appetite suppressants or stimulants, anti-wasting disorder agents, anti-bulimic agents, anti-anorexia nervosa agents, brain injury agents, heart attack agents, adenosine, adenosine releasing agents, adenosine receptor stimulating agents, sun screens, emollients, skin temperature lowering agents, radioactive phosphorescent or fluorescent contrast diagnostic or imaging agents, libido altering agents, bile acids, laxatives, anti-diarrheic agents, skin renewal agents or hair growth agents.

195. The method of claim 194, wherein the composition comprises the first agent(s), a hormone(s) having estrogen or progesterone activity, a diuretic(s) and an analgesic or anti-inflammatory agent(s).

196. A foodstuff, comprising the composition of claim 95, and other edible ingredients.

197. The foodstuff of claim 196, selected from milk, juices, cereals, energy bars, chewing gum, cookies, candy, crackers, drinks, cakes, pasta, and salad dressing, vegetables, meats or fruits.

198. The product of claim 197, wherein the foodstuff comprises infant formula, milk, baby food, cookies, or crackers.

199. A kit for preparation of a foodstuff, comprising in separate containers
the first agent of claim 95;
foodstuff ingredients; and
instructions for combining the foodstuff ingredients with the first agent and other non-provided ingredients, and for preparation of the foodstuff.

200. The kit of claim 199, wherein the ingredients and the first agent are provided in a foodstuff composition.

201. The kit of claim 199, further comprising in a separate container a second agent selected from analgesics, anti-pre-menstrual syndrome agents, anti-menopausal agents, anti-aging agents, anti-anxiolytic agents, other mood controlling agents, anti-depressants, anti-bipolar mood disorder agents, anti-schizophrenic agents, anti-cancer agents, aggression controlling agents, anti-attention deficit disorder agents, anti-delirium agents, anti-compulsive obsessive disorder agents, anti-aggression agents, anti-seizure agents, alkaloids, blood pressure controlling agents, hormones, anti-inflammatory agents, muscle relaxants, steroids, soporific agents, anti-ischemic agents, anti-arrhythmic agents, contraceptives, vitamins, hormonal agents, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents or hair growth agents.

202. The kit of claim 199, further comprising in a separate container a surfactant selected from surfactant protein A, surfactant protein B, surfactant protein C, surfactant protein D and surfactant protein fragments thereof, non-dipalmitoyl disaturated phosphatidylcholine, dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholin, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate, artificial lamellar bodies vehicles for surfactant components, omega-3 fatty acids, polyenic acid, polyenoic acid, lecithin, palmitic acid, non-ionic ethylene and/or propylene oxide block copolymers, polyoxypropylene, polyoxyethylene, poly (vinyl amine) with dextran and/or alkanoyl side chains, Brij 35®, Triton X-100®, ALEC®, Exosurf®, Survant® or Atovaquone®.
